(19) United States
(12) Patent Application Publication
(10) Pub. No.: US 2005/0020982 A1
(43) Pub. Date: Jan. 27, 2005

(54) Medical device security band

(76) Inventor: Sheila Shaw, Tracy, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 10/894,135

(22) Filed: Jul. 19, 2004

Related U.S. Application Data
(63) Continuation-in-part of application No. 10/690,289, filed on Oct. 21, 2003.
(60) Provisional application No. 60/488,958, filed on Jul. 22, 2003.

(51) Int. Cl. A61M 5/32

(52) U.S. Cl. 604/179

(57) ABSTRACT

The present invention provides a medical device security band including an elastic band and a medical device retaining pocket positioned along a portion of the elastic band. The elastic band is not detachable and the medical device retaining pocket is able to accept a medical device. The present invention also includes methods of use.

### CROSS REFERENCE TO RELATED APPLICATIONS

The present application is a Continuation-in-Part of U.S. patent application Ser. No. 10/690,289 filed on Oct. 21, 2003, entitled “Peritoneal Dialysis Security Band” and naming Sheila Shaw as inventor, which claims benefit of priority to U.S. Provisional Patent Application Ser. No. 60/488,958 entitled “Peritoneal Dialysis Security Band”, filed Jul. 22, 2003, and naming Sheila Shaw as inventor, both of which are incorporated by reference in their entirety.

### TECHNICAL FIELD

The present invention relates to apparatuses that secure medical devices, and more particularly relates to a non-detachable medical device security band for retaining a medical device such as a diagnostic or therapeutic medical device.

### BACKGROUND OF THE INVENTION

Portable medical devices able to detect, monitor or treat a variety of medical conditions are currently available. Increasingly, patients are able to carry these medical devices on their person for quick and convenient detection or treatment of a condition.

Diabetes is a medical condition where the body does not sufficiently produce or utilize insulin. Insulin is a hormone that is needed to convert sugar, starches and other food into energy needed for daily life. A patient with a fasting blood glucose level of 126 mg/dL or greater has diabetes.

Diabetic patients regularly measure their blood glucose level to determine whether the administration of insulin is required. Administration of insulin traditionally involved injection using a syringe. However as technology has progressed, glucose monitors have been designed in fluid communication with the blood stream allowing easy and frequent detection of blood glucose levels and insulin pumps are now being used, which may or may not be integrated with a glucose monitor, to provide a continuous or a bolus flow of insulin. Thus insulin pumps have reduced the requirement of injections by syringe, however the insulin pump or glucose monitor must be kept in close proximity of the patient since the device is fluidly attached to the patient.

Peritoneal dialysis is a technique intended to mimic the effect of the kidneys and involves the removal of soluble waste substances from the body by a transfer process across the peritoneum. This procedure, often referred to as an exchange, includes introducing a dialysis solution into the peritoneum of an individual, allowing the solution to collect soluble waste and draining the waste solution. Fluids are transferred using a surgically implanted device called a catheter. Without proper care, the catheter can become dislodged resulting in infection and potential death.

Since the catheter has an external portion, the patient must store the external portion of the catheter along the body. Traditionally, a patient would adhesively tape the external portion of the catheter to the abdomen. However, this technique can be uncomfortable to the patient and frequent removal and re-taping of the catheter often causes irritation.

Several U.S. patents offer an alternative to taping the catheter to the abdomen. These devices frequently incorporate a detachable belt. Generally the opposing ends of a detachable belt are reversibly fastened using a fastening means or fastening structure. Examples include hooks, snaps, buttons, buckles, VELCRO (R) the like. However, there are multiple disadvantages to a detachable peritoneal dialysis belt. First, a fastening structure may lead to binding, pinching, scratching, or irritation of the skin. Second, the belt is typically fastened after retention of a catheter, which may lead to an uncomfortable pulling or tugging on the catheter while attempting to fasten the ends. Third, since the belt may be reversibly fastened there is a risk of the garment coming undone. Once the garment comes undone, the catheter may be exposed to a risk of becoming dislodged, pulled, or stressed. Fourth, if the fastening structure is constructed from metal, the patient risks exposure to rust after machine washing the device or while the device rests against the moisture of the patient's body.

U.S. Pat. No. 5,468,229 discloses a reversibly securable peritoneal dialysis belt having an aperture for receiving and orientating the protruding portion of a catheter towards a plurality of holders along one portion of the belt. Referring to column 2, line 25, this device requires the exterior portion of the catheter be inserted through an aperture and guided through the plurality of holders thereby securing and supporting the entire external portion of the catheter. Referring to column 3, line 1, the securing means may be a hook and loop, snap or button. A disadvantage of this device is that feeding the catheter through the aperture then through the plurality of holders involves significant manipulation of the catheter. This manipulation may lead to pulling the catheter causing discomfort. Secondly, the catheter must be fed through the aperture and likely through the holders prior to attaching the ends of the belt. Therefore fastening the ends may include pulling or tugging the retained catheter causing discomfort. Thirdly, a fastening structure may lead to binding, pinching, scratching, or irritation of the skin. Fourthly, because the belt may be reversibly fastened, there is a risk of the garment coming undone.

U.S. Pat. No. 6,436,074 discloses a garment for securing and exposing a peritoneal dialysis catheter and catheter exit site including a slip resistant torso belt, a pouch into which the end of the disconnected catheter can be placed, and a block of foam rubber permanently attached inside the belt. Column 3, line 26 describes the belt of the device as consisting of an elastic strap which can be fastened into a torso belt using Velcro ® fasteners. Column 3, line 35 describes the pouch as having an opening once at its top along the length of the pouch and again through a slit at the exit site covering. Therefore this device includes a detachable belt and a pocket containing an entry aperture and an exit aperture along the top. Use of this device requires the catheter be fed through two apertures, which may lead to pulling of the catheter and discomfort. In addition, the belt has detachable ends and therefore the fastening structure may lead to binding, pinching, scratching, or irritation of the skin. Also, because the belt may be reversibly fastened, there is a risk of the garment coming undone.

U.S. Pat. No. 5,853,396 discloses a tuck-away belt for peritoneal dialysis patients including a flexible elongated pocket envelope having two panels and a flexible elastic band narrower than the envelope pocket and having a hook and loop type attachment at one end and a D-ring at the opposite end through which the elastic band is pulled through to attach itself. Thus this device includes a flexible envelope attached to a reversibly attachable narrow belt. This device includes a hook and loop fastener. A hook and loop type fastener may lead to binding, pinching, scratching, or irritation of the skin. In addition since a hook and loop type fastener may be reversibly fastened, there is a risk of the garment coming undone.

U.S. Pat. No. 6,126,639 discloses a continuous ambulatory peritoneal dialysis catheter support under garment including a belt of expandable material, a pocket portion, and a means for releasably attaching a first and second end. Referring to column 2, line 30, the patient inserts the catheter into the pocket portion, wraps the ends of the garment around their waist or hips, and attaches the ends. Referring to column 3, line 58, the releasable attachment means or suitable securing means may utilize hook and loop, buttons, snaps, buckles, re-usable tape, and the like. Thus, this dialysis support undergarment includes a detachable belt with a securing means or fastening structure. The securing means may therefore lead to binding, pinching, scratching, or irritation of the skin. Moreover since the securing means is reversibly fastened, there is a risk of the garment coming undone.

Therefore there is a need for a security device for patients carrying medical devices such as glucose monitors, insulin pumps, catheters and the like that reduces binding, pinching, scratching, or irritation of the skin that may occur with traditionally catheter belts and that have a reduced the risk of the garment coming undone.

### BRIEF SUMMARY OF THE INVENTION

It is the object of the present invention to provide a medical device security band that reduces, minimizes or eliminates the binding, pinching, scratching, or irritation of the skin that can occur with a catheter belt having detachable ends. It also the object of the present invention to provide a medical device security band that reduces, minimizes or eliminates the risk of the garment coming undone.

Specifically, the present invention provides a medical device security band including a medical device retaining pocket located along a portion of the elastic band. The elastic band is not detachable and the medical device retaining pocket is able to accept an external medical device for the detection, treatment or maintenance of a medical condition.

The present invention also provides a method of retaining the external portion of a medical device such as an insulin pump or an implanted peritoneal dialysis catheter including placing the medical device security band of the present invention about the body such as the arm, leg, waist or hips of an individual and inserting the medical device within the medical device retaining pocket. The method may also include placing the medical device security band about the body such as the arm, leg, waist or hips prior to inserting the medical device in the medical device retaining pocket.

### BRIEF DESCRIPTION OF THE DRAWINGS

FIG. 1 depicts a medical device security band 10 for use with peritoneal dialysis. The depicted elastic band 11 is a continuous singular band having a medical device retaining pocket 12 positioned along a portion of the inside surface of the elastic band 11. FIG. 1A is an elevated rear right view depicting the medical device retaining pocket 12 along the inner surface of the elastic band and FIG. 1B is an elevated front right view with the hidden medical device retaining pocket 12 shown in dotted lines.

FIG. 2 depicts a front plan view of a portion of the inside surface of a medical device security band 10 showing the medical device retaining pocket 12 positioned along the inside of the elastic band 11.

FIG. 3 depicts a front view of a medical device security band 10 adapted for use with peritoneal dialysis. The medical device security band 10 is shown positioned about the waist of an individual. The medical device retaining pocket 12 is depicted as dotted lines and is positioned along the inside surface of the elastic band 11. A catheter is shown in dotted lines and is positioned within the medical device retaining pocket 12.

FIG. 4 depicts a front plan view of a medical device security band 10 positioned about the hips or waist of an individual with the elastic band 11 folded forward to reveal a catheter partially positioned in the medical device retaining pocket 12.

FIG. 5 is an elevated rear right view of a security band 10 adapted for use with an insulin pump. The medical device retaining pocket 12 is positioned along the inner front of the elastic band 11 and can be accessed along the patient's left side.

### DETAILED DESCRIPTION

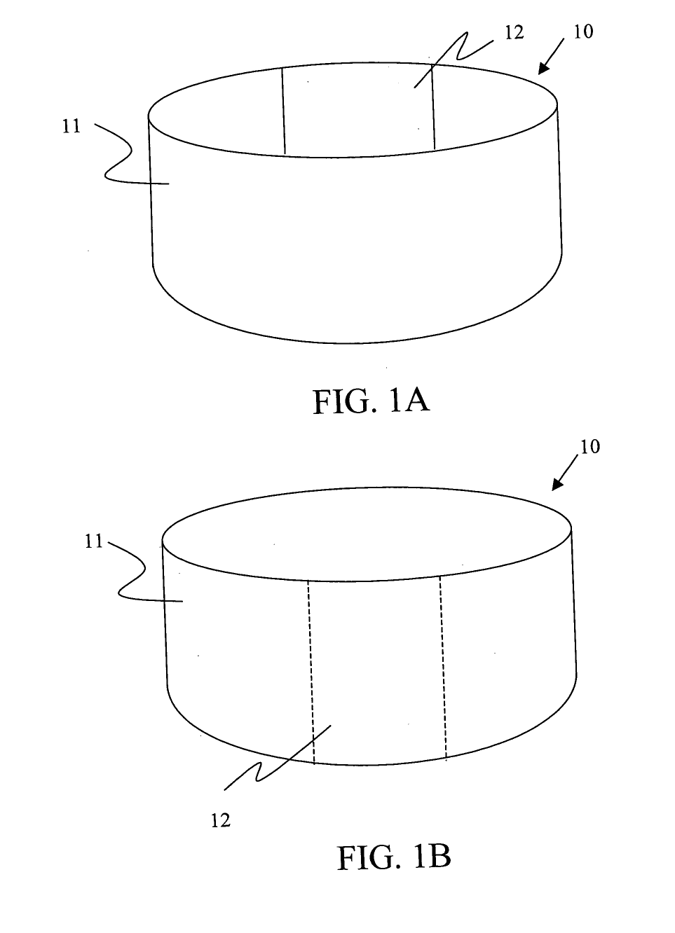

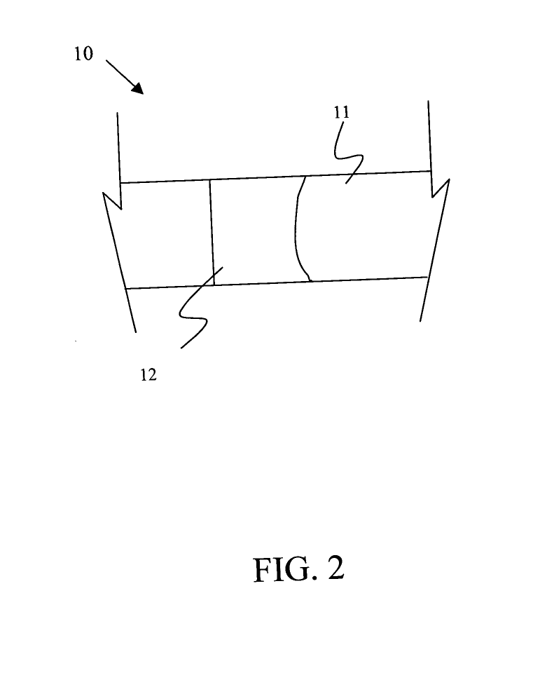

Referring to FIGS. 1-5, the disclosed medical device security band 10 includes an elastic band 11 and a medical device retaining pocket 12. The medical device retaining pocket 12 is able to accept or retain a variety of medical devices for the detection, treatment or maintenance of a medical condition such as an insulin pump or the external portion of a catheter. The retaining pocket 12 may be affixed to or located along a portion of the elastic band 11. Preferably the medical device retaining pocket 12 is positioned along a portion of the inside surface of the elastic band 11 however the catheter retaining pocket 12 may be positioned along a portion of the outside surface of the elastic band 11. As will be realized, the present device provides significant advantages over previously described catheter belts for peritoneal dialysis patients and provides benefits to patients having other medical conditions where previously described catheter belts can not be used, have disadvantages or are not preferred. For example, the medical device security band 10 of the present invention reduces or eliminates binding, pinching, scratching, or irritation of the skin; reduces or eliminates the pulling or tugging of a medical device that has an invasive portion such as but not limited to a catheter or an insulin delivery tube; and reduces or eliminates the likelihood of the garment coming undone. Among these and other benefits, the medical device security band 10 may provide the patient with additional back support and may be machine washable.

The security band 10 of present invention may be utilized to secure a variety of medical devices. Medical devices that may be secured or retained may include those that are used in the detection, treatment or maintenance of a medical condition. For example, the medical device may detect or monitor the presence or amount of an analyte in a bodily fluid or may measure bodily conditions such as heart rate, temperature and the like. Non-limiting examples of analytes that may be detected or monitored are blood glucose, cholesterol and the like. Typically the medical device is closely associated with the patient such as a portion of the device or system in contact with or inserted within the patient.

Non-limiting examples of medical devices that may be used with the present invention that detect an analyte are insulin pumps or glucose monitoring devices. These devices typically involve or include a cannula, needle or inner tube portion fluidly connected to a monitor or pump. A transfer tube, also referred to herein as an insulin transfer tube, allows fluid to pass between the body and the medical device. Blood or a bodily fluid may pass from the body to the monitor or pump and a medication such as insulin may pass from the pump or monitor to the body.

Insulin pumps such as but not limited to the 500 series offered by Medtronic MiniMed provide a combination of glucose monitoring and insulin treatment and can be retained with the present invention. These insulin pumps are generally about the size of a beeper and are connected to a cannula, which is placed under the skin, via a plastic tube. The insulin pump allows the patient to regularly monitor glucose levels and provide a continuous delivery of insulin. Because the pump is, in essence, attached to the body, the present invention is able to retain the pump at a convenient or comfortable location.

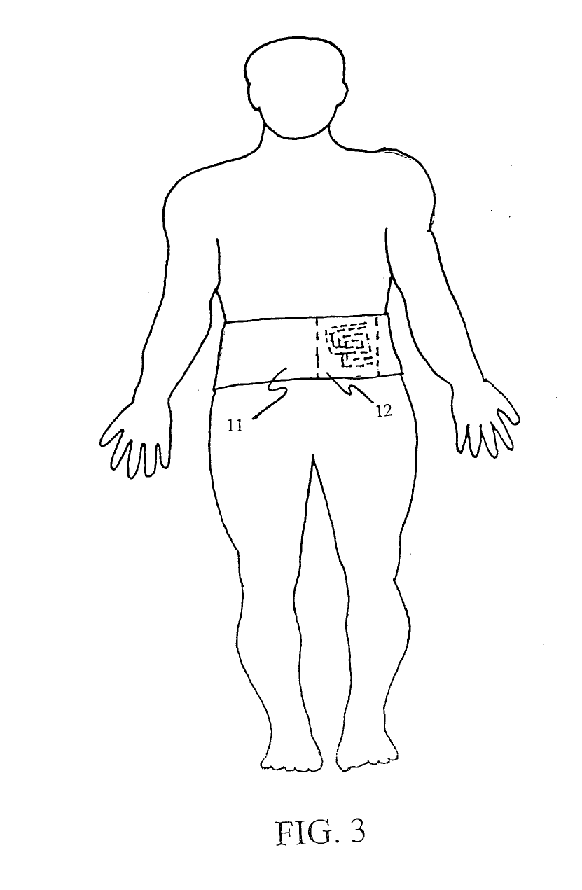

Referring to FIGS. 3-5, the medical device security band 10 may be worn about the waist or hips. In these embodiments, the security band 10 may be placed about the patient's waist by stretching the elastic band 11, inserting the patient's legs through the elastic band 11, sliding the security band 10 upwards along and about the patient's legs ending at about the waist or hips of the patient and releasing the elastic band 11. The medical device retaining pocket 12 may be exposed for insertion of a medical device such as a catheter or insulin pump by stretching the elastic band 11 generally outward or folding the elastic band 11 generally forward. The medical device security band 10 may be aligned such that the medical device retaining pocket 12 is oriented near the site of treatment such as the exterior portion of the catheter or insulin delivery tube. However, alignment of the security band 10 may differ depending on the medical device retained and the patient's comfort.

The medical device security band 10 may be offered in a variety of sizes to accommodate a variety of body parts such as arms, legs, chests or waistlines or may be offered in a one-size-fits-all configuration. The size may generally correspond to the size of the elastic band 11. As a non-limiting example the medical device security band 10 may be provided in extra small (XS), small (S), medium (M) and large (L) sizes. More specifically, when used about the waist, an extra small may be offered to accommodate a waist size from about sixteen inches to about twenty-six inches, a small from about twenty-six inches to about thirty-six inches, a medium from about thirty-six inches to about forty six inches, and a large from about forty-six inches to about fifty-six inches. Alternatively, a one-size-fits-all security band 10 may accommodate a waist size from about sixteen to about fifty-six inches or more. However the present invention encompasses larger and smaller sizes and the size ranges may differ depending on the portion or part of the body or limb for which the device is worn and is not meant as a limiting feature.

The elastic band 11 is nondetachable and may be worn generally about waist or hips of the patient such that a catheter may be retained in the medical device retaining pocket 12. The elastic band 11 may be provided in a variety of sizes allowing the medical device security band 10 to fit a variety of waistlines. The vertical width of the elastic band 11 may be variable and should be sufficiently wide to facilitate the medical device retaining pocket 12 or facilitate the attachment of a medical device retaining pocket 12. The vertical width of the elastic band 11 may therefore be vertically wider, vertically shorter or have the same vertical width of the medical device retaining pocket 12. As general guidance, the vertical width may be from about one half inch to about ten inches or more. When used to retain a catheter, the preferred width may be from about three inches to about nine inches. As can now be envisioned, the width of the elastic band 11 may vary widely depending on the size of the medical device retaining pocket 12, which itself may vary in size depending on the medical device. Thus the present invention envisions and teaches the security band 10 may be provided in any size desired by the patient.

The elastic band 11 is a band with elastic features and thus may be constructed entirely from an elastic material or may be constructed in part from an elastic material and in part from an inelastic material. The elastic material allows the medical device security band 10 to stretch and accommodate a variety of waistlines or body parts. Examples of appropriate elastic materials or fabrics include those used in the garment industry such as but not limited to spandex, Lycra and elastic rubber. In addition, the elastic band 11 may utilize a variety of inelastic materials or fabrics used in the garment industry such as but not limited to cotton, cotton blend, nylon, polyester, and inelastic rubber. The elastic band 11 may be constructed from a blend of materials. Preferably the elastic band 11 and medical device retaining pocket 12 are constructed from a material including 88% polyester and 12% Spandex. Transparent materials such as rigid or non-rigid transparent plastics may be utilized in whole or in part so that the medical device such as a detection device or therapeutic device may be visually read or adjusted while retained in the retaining pocket 12. Materials may be used alone or in combination with one another and are preferably machine washable and breathable.

In one configuration, the elastic band 11 is constructed entirely from a machine washable elastic fabric. The elastic fabric is cut to the desired size and the opposing ends are sewn together or irreversibly affixed to form a non-detachable elastic band 11.

In another configuration, the elastic band 11 is constructed from two or more elastic portions in vertical alignment such as above or below one another with at least one inelastic portion. The inelastic portion may function to attach the elastic portions together and may limit the amount that the elastic band 11 may stretch. For example, an elastic rubber portion may extend horizontally and be vertically aligned such as above or below one or more additional elastic rubber portions. An inelastic polyester portion may be vertically aligned between and attached to the elastic rubber portions. There may exist spaces or gaps within the inelastic polyester portion where no material or fabric exists thereby allowing the elastic portion to stretch horizontally.

In another configuration the elastic band 11 is constructed from three or more elastic portions in vertical alignment such that the top most, bottom most or both top most and bottom most stretch less or are sized smaller than the inner elastic portions. Thus, a security band 10 may stretch to a lesser degree at about the top or bottom than the middle.

In another configuration, the elastic band 11 is constructed from an elastic material in horizontal alignment with an inelastic material creating a region that stretches horizontally and a region that does not substantially stretch horizontally. In this configuration distinct panels may be provided that either substantially stretch or do not substantially stretch and therefore control the adjustment size of the security band 10. For example, the opposing ends of a lycra panel may be sewn to a panel of a cotton blend. In this example the lycra allows the elastic band 11 to stretch and therefore adjust to a larger waistline while the cotton blend limits the amount of stretching of the elastic band 11 thereby limiting the adjustment of the elastic band 11.

In another configuration, the elastic belt 11 is constructed in part from an elastic material and in part from an inelastic material where the materials are combined in a single panel or region. For example, an elastic rubber may be encased, surrounded or interweaved with an inelastic polyester fabric. In this example the polyester fabric may be bunched up until the elastic rubber is stretched. At this point the polyester fabric becomes taught and the elastic band 11 has reached its limit as to size.

Referring to FIG. 3, the medical device retaining pocket 12 may function to retain a medical device such as a catheter to the elastic band 11. The medical device retaining pocket 12 is shown positioned along a portion of the inside of the elastic band 11. In this embodiment medical device retaining pocket 12 should be sufficiently large that the external portion of the catheter may be placed substantially within the medical device pocket 12. For example, the medical device retaining pocket 12 may be about one to ten inches horizontally long by about one to ten inches vertically wide however any dimensions allowing the retention of a catheter or the particular medical device are sufficient.

The medical device retaining pocket 12 may be constructed from a variety of elastic and inelastic materials such as spandex, lycra, elastic rubber, cotton, cotton blend, nylon, polyester, and inelastic rubber or any combination thereof. Preferably the medical device retaining pocket 12 is constructed from at least in part an elastic material to further assist the retention of the medical device. An elastic material may assist in reducing movement of the retained medical device.

The medical device retaining pocket 12 may be formed separately then affixed to the elastic band 11. Alternatively the catheter retaining pocket 12 may be formed upon sewing or affixing an elastic or inelastic material or fabric to the elastic band 11 resulting in a pocket or pouch able to accept a medical device. As another non-limiting example, the medical device retaining pocket 12 may be formed by overlapping the ends of the elastic band 11 and sewing along three of four perimeter sides or the like of the overlapping portion thereby forming a pocket able to accept a medical device.

The medical device retaining pocket 12 may include a closing structure able to substantially reversibly close the pocket 12 such that the majority of the opening or access aperture to the pocket 12 may be opened and closed. This may be provided in a variety of configurations such as but not limited to the utilization of a zipper, button, hook and loop, a drawstring, Velcro (R) and the like. Partially or substantially closing the pocket 12 may assist in retaining the medical device. A portion of the pocket 12 may be left open to permit access of a tube or the like however, in other embodiments the transfer tube is threaded through an aperture in a side wall of the pocket 12 and the pocket access aperture is able to completely close.

The present invention also includes a method of retaining the external portion of a medical device such as a catheter or an insulin pump including placing the medical device security band 10 of the present invention about the body such as the arm, leg, chest, waist or hips of an individual and inserting the medical device such as the external portion of the implanted catheter or insulin pump in the medical device retaining pocket 12. The medical device such as the catheter or insulin pump may be placed in the medical device retaining pocket 12 after positioning the elastic band 11 about the body such as the waist or hips.

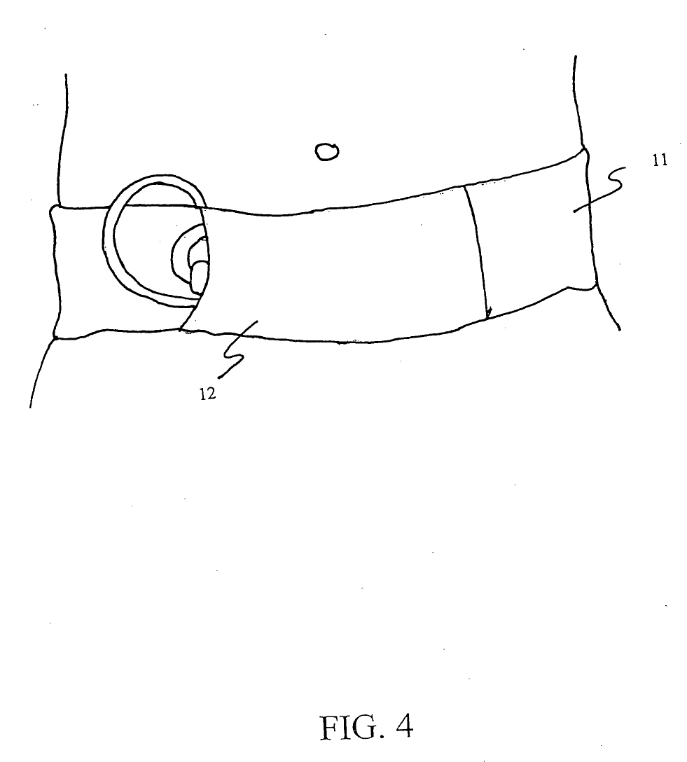

When utilized for dialysis and once the patient must perform the dialysis procedure, the catheter may be easily accessed. The elastic band 11 may be stretched generally outward or folded generally forward as shown in FIG. 4, allowing access to the medical device retaining pocket 12. The catheter may then be removed from the medical device retaining pocket 12 and used according to the physician's instructions. Likewise, reinsertion of the catheter into the medical device retaining pocket 12 may involve stretching the elastic band 11 generally outward or folding the elastic band 11 generally forward exposing the medical device retaining pocket 12 and reinserting the catheter into the medical device retaining pocket 12.

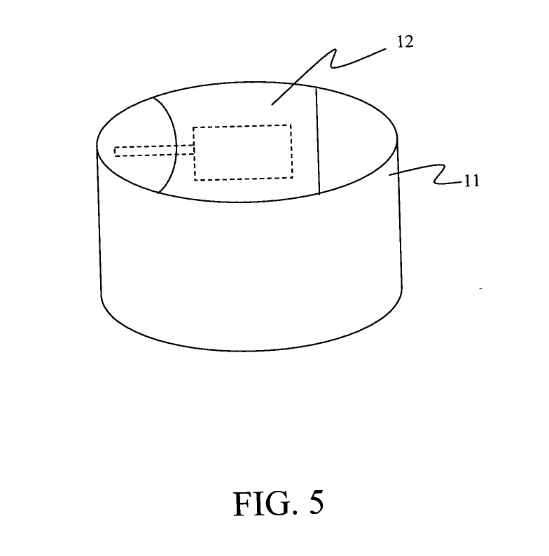

Referring to FIG. 5, the present invention may also be utilized for medical devices for the detection, treatment or maintenance of medical conditions such as diabetes. A glucose monitor or insulin pump may be placed within the medical device retaining pocket 12. A tube such as an insulin transfer tube or a tube for the transfer of blood for the detection of glucose extends from the medical device to the point of contact with the patient. Excess tubing may also be placed in the medical device retaining pocket 12. The medical device may be removed from the security band to visually read the medical device or when the elastic band 11 and/or medical device retaining pocket 12 is constructed in part from a transparent material, the medical device may be viewed or adjusted while remaining in the medical device retaining pocket 12.

All documents including patent documents referred to in this application are incorporated by reference in their entirety for all purposes to the same extent as if each individual publication were individually incorporated by reference.

1. A medical device security band comprising:
a) a elastic band, wherein said elastic band is not detachable; and
b) a medical device retaining pocket able to retain a medical device, wherein said medical device retaining pocket is positioned along a portion of said elastic band.

2. The medical device security band according to claim 1, wherein said elastic band comprises an inner surface and an outer surface, further wherein said medical device retaining pocket is positioned along a portion of said inner surface.

3. The medical device security band according to claim 1, wherein said elastic band is constructed at least in part from an elastic material selected from the group consisting of spandex, lycra and elastic rubber.

4. The medical device security band according to claim 1, wherein said elastic band comprises a middle portion able to stretch further than a top portion or a bottom portion.

5. The medical device security band according to claim 1, wherein said elastic band is constructed at least in part from an inelastic material.

6. The medical device security band according to claim 5, wherein said inelastic material is selected from the group consisting of cotton, nylon, polyester and inelastic rubber.

7. The medical device security band according to claim 1, wherein said elastic band is foldable.

8. The medical device security band according to claim 1, wherein said elastic band is constructed from a machine washable material.

9. The medical device security band according to claim 1, wherein said elastic band has a vertical width from about one inch to about nine inches.

10. The medical device security band according to claim 1, wherein a portion of said elastic band is transparent, further wherein said transparent portion is in alignment with said medical device retaining pocket allowing said medical device to be viewed while retained.

11. The medical device security band according to claim 1, wherein said medical device retaining pocket has a vertical width from about one inch to about nine inches.

12. The medical device security band according to claim 11, wherein said medical device retaining pocket further comprises a closing structure able to reversibly and substantially close said medical device retaining pocket.

14. The medical device security band according to claim 13, wherein said closing structure is selected from the group consisting of a snap, a zipper, a hook and loop, a drawstring and Velcro.

15. The medical device security band according to claim 1, wherein a portion of said medical device retaining pocket is transparent, further wherein said medical device may be viewed through said transparent portion while said medical device is retained.

16. The medical device security band according to claim 1, wherein said medical device is selected from the group consisting of a catheter, a glucose monitor or an insulin pump.
